(19) United States
(12) Patent Application Publication
(10) Pub. No.: US 2007/0196313 A1
(43) Pub. Date: Aug. 23, 2007

(54) Cleansing Compositions Containing Film

(76) Inventors: Diana Scala, Hillsborough, NJ (US); Liem Vu, Berkeley Heights, NJ (US); Thomas J. Boyd, Metuchen, NJ (US); Regina Hourigan, Metuchen, NJ (US); Sayed Ibrahim, Somerset, NJ (US); Ariel Haskel, East Brunswick, NJ (US)

Correspondence Address: COLGATE-PALMOLIVE COMPANY, 909 RIVER ROAD, PISCATAWAY, NJ, 08855, US

(21) Appl. No.: 11/465,525

(22) Filed: Aug. 18, 2006

Related U.S. Application Data
(60) Provisional application No. 60/709,402, filed on Aug. 18, 2005.

(51) Int. Cl. A61K 8/73

(52) U.S. Cl. 424/70.13

(57) ABSTRACT

A composition comprising a cleanser base and a plurality of film pieces, wherein the composition has a moisture content of about 22% or less. Also, a method of making a composition, comprising the steps of combining pieces of film with a cleansing material to make a cleansing mixture, and forming the mixture into a product of desired shape and size. Also, A method of delivering an active material during cleansing, comprising cleansing with water and a composition, wherein the composition comprises a plurality of film pieces, said film pieces comprising an active material and a polymer selected from water-soluble polymers, water-dispersible polymers, and combinations thereof.

### CROSS REFERENCE TO RELATED APPLICATION

This application claims priority to U.S. Provisional Patent Application Ser. No. 60/709,402, filed on Aug. 18, 2005, which is incorporated herein by reference.

### BACKGROUND OF THE INVENTION

Cleansing compositions, including cleansing bars, are widely used for personal cleansing. Among desirable attributes for such compositions is aesthetic appeal, which may include an appearance suggestive of multiple beneficial properties the product may provide during cleansing. Certain natural products, such as leaves or flowers, may be included to suggest nature and freshness, but may bleed or discolor in the product. Natural products may also have trouble surviving the manufacturing process for the compositions and thus require special manufacturing procedures. Other materials, such as oatmeal or beads, may be included for exfoliation or other desirable properties, but offer limited visual appeal.

### SUMMARY OF THE INVENTION

A composition comprising a cleanser base and a plurality of film pieces, wherein the composition has a moisture content of about 22% or less. Also, a method of making a composition, comprising the steps of combining pieces of film with a cleansing material to make a cleansing mixture, and forming the mixture into a product of desired shape and size. Also, A method of delivering an active material during cleansing, comprising cleansing with water and a composition, wherein the composition comprises a plurality of film pieces, said film pieces comprising an active material and a polymer selected from water-soluble polymers, water-dispersible polymers, and combinations thereof.

### DETAILED DESCRIPTION OF THE INVENTION

As used throughout, ranges are used as a shorthand for describing each and every value that is within the range. Any value within the range can be selected as the terminus of the range. The following description of the preferred embodiment(s) is merely exemplary in nature and is in no way intended to limit the invention, its application, or uses.

The compositions of the invention comprise a cleanser base (e.g., soap) (also referred to as “cleansing material”) containing film in desired fragment sizes and shapes. The film fragments or pieces may be of a desired thickness. The film preferably includes a film-forming material and a functional material.

In preferred form, the composition of the invention has a moisture content of about 22% or less. In certain embodiments, the composition may be in the form of a cleansing bar, for example, a soap bar.

In certain embodiments, the pieces of film may be visible and may have an irregular or regular, repeating shape and/or color to lend to visual appeal of the product. The film may contain a functional material, such as a colorant or an active ingredient that provides benefits (e.g., cleansing, sudsing, moisturizing) during cleansing. The composition may have two or more kinds of film pieces that differ, for example, in color, shape, size, film-forming materials, and/or functional material content.

According to various embodiments of the invention, the film comprises a film-forming material. In certain embodiments, the film-forming material may be a film-forming polymer. In certain embodiments, the film comprises a water-soluble or water-dispersible polymer. The film dissolves or breaks up when the composition is used in cleansing with water. The film may release an active material, such as a fragrance, essential oil, moisturizer, or other active, when the composition is used in cleansing. The film may also include a water-insoluble polymer and/or a water-soluble filler material.

In another aspect, the invention provides a method of delivering an active material during cleansing with a composition. The active material is included in pieces of polymeric film contained in the composition, and is released from the film pieces during cleansing. Release of the active material is affected by selection of film thickness, size of film pieces, concentration of active in the film, concentration of the film in the composition, and film composition (e.g., the combination of film-forming materials and, if used, fillers in forming the film).

Further provided is an extrusion method for forming a cleansing product in which pieces of polymeric film are combined with a composition material and the mixture of pieces of polymeric film and cleansing material is extruded and formed into cleansing products containing the pieces of polymeric film. Depending on the film composition, the pieces may be added in an amalgamator or into the extruder to be combined with the cleansing material. Preferably, the film-forming material should have a glass transition temperature of at least about 37° C., for example at least about 50° C. or at least about 60° C. so that the film pieces will remain relatively intact in the product.

The invention still further provides a method of making a cleansing product in which pieces of film and composition material are combined in an amalgamator to form a mixture, the mixture is passed through a typical toilet soap line that includes, e.g., a refiner, mill, and vacuum plodder for further refining, homogenizing, and forming into a billet. The billet is cut and pressed into products of desired size and shape.

In yet another embodiment of the invention, pieces of film and composition material are combined in a refiner, mill, or vacuum plodder, extruded as a billet, and cut into a desired size and shape.

As referred to herein, a “film” is a material having a substantially lamellar structure. A “lamellar” structure has, or is capable of having, a size in one or two dimensions (e.g., the x- or y-dimensions) that is substantially greater than the thickness of the structure in a third dimension (e.g., the z-direction). Lamellar structures among those useful herein include those that are substantially planar, layered, or lamelliform. In one embodiment, the lamellar structure is substantially planar, having a size in both the x- and y-dimensions that is substantially greater than the z-direction. In other embodiments, the lamellar structure is non-planar. In one embodiment, a film of this intention comprises a substantially continuous surface that can appear as a substantially flat surface, although in some embodiments the film may be deformed. In such embodiments, the film can have any of a number of shapes, including having a smooth curved surface.

In certain embodiments, the film comprises at least one film-forming polymer or a mixture of film-forming polymers. Preferably, the film-forming polymer has a glass transition temperature of preferably at least about 37° C., for example at least about 50° C. or at least about 60° C. Other polymeric materials that have lower glass transition temperatures may also be included, so long as the film is sufficiently durable to come through the process of making the composition substantially intact. That is, some degradation of film pieces may take place during mixing, extruding, and composition forming, but the pieces will retain a desired size overall.

In certain embodiments, the polymer or polymers preferably include at least one water-soluble or water-dispersible polymer. A sufficiently durable polymer or combination of polymers may be selected from, for example, poly(vinyl pyrrolidone) [PVP], which may preferably have a weight average molecular weight of about 100,000 or more and up to about 1.5 million, poly(vinyl alcohol) [PVA], poly(vinyl pyrrolidone/vinyl acetate) copolymers such as Kollidon® VA64 (available from BASF, 60:40 by weight vinyl pyrrolidone:vinyl acetate, Mw reported as 45,000 to 70,000) and Plasdone® S630 PVP (available from International Specialty Products, Wayne, N.J., 60:40 by weight vinyl pyrrolidone : vinyl acetate), ethylene oxide graft copolymers of PVA such as Kollicoat® IR (available from BASF, 75% by weight PVA, 25% by weight polyethylene glycol graft, Mw reported to be about 45,000), water-soluble cellulose derivatives including, without limitation, methyl cellulose, ethyl methyl cellulose, hydroxylated and carboxylated celluloses including hydroxyalkyl cellulose and hydroxyalkyl alkyl cellulose polymers such as hydroxypropyl methyl cellulose (HPMC), hydroxyethyl methyl cellulose hydroxypropyl cellulose (HPC), hydroxyethyl cellulose (HEC), and, carboxymethyl cellulose (CMC); and polyoxyethylene polymers and copolymers. In certain preferred embodiments, the film-forming polymer includes one or more polymers selected from hydroxyalkyl alkyl celluloses, hydroxyalkyl celluloses, methyl celluloses, and combinations of these.

The film may include a water-soluble filler. Suitable examples of water-soluble fillers include, without limitation, natural gums such as sodium alginate, carrageenan, xantham gum, gum acacia, arabic gum, guar gum, pullulan, agar, chitin, chitosan, pectin, karaya gum, zein, hordein, gliadin, locust bean gum, tragacantha and other polysaccharides; starches such as maltodextrin, amylose, high amylose starch, corn starch, potato starch, rice starch, tapioca starch, pea starch, sweet potato starch, barley starch, wheat starch, waxy corn starch, modified starches (e.g. hydroxypropylated high amylose starch), dextrin, levan, elsinan and gluten; and proteins such as collagen, whey protein isolate, casein, milk protein, soy protein, and gelatin. The film may further include dispersible or swellable filler such as modified starch, alginate esters, and divalent or multivalent ion salts of alginates.

Gums, PVP, and PVA are preferably used in the film only in conjunction with another film-forming polymer or filler. Gums, PVP, and PVA are preferably included in the film along with, for example, cornstarch and/or HPMC. One preferred combination for a composition containing soap is a film including HPMC and cornstarch. Other preferred polymers include those known (like HPMC) to enhance and stabilize lather volume and provide silky, smooth sensation.

In various embodiments, it is desirable to include at least a small amount of a gum, e.g. carageenan, and/or a polyoxyethylene polymer or graft copolymer to attain desired film properties such as tensile strength. The gum or polyoxyethylene polymer or copolymer may be included in an amount of at least about 0.01 wt.%, preferably at least about 0.05 wt. %, more preferably at least about 0.1 wt.%, and up to about 5 wt.%, preferably up to about 1 wt.% of the film.

In various embodiments, it may be preferred to include a partially water-soluble polymer in addition to, or instead of, a water-insoluble polymer. Examples of partially water-soluble polymers include, for example, poly(vinyl pyrrolidone/vinyl acetate) copolymers in which the weight percent of vinyl pyrrolidone monomer is less than about 50%, such as Luviskol VA 37 E (available from BASF, 30:70 by weight vinyl pyrrolidone : vinyl acetate), and physical mixtures of PVP and PVAc, such as Kollidon® SR, which is 80% by weight PVAc and 19% by weight PVP (1% by weight stabilizers) available from BASF.

The film may include one or more water-insoluble polymers. Useful water-insoluble polymers include, for example, acrylic copolymers (which may in the form of acrylic copolymer dispersions), crosslinked poly(vinyl pyrrolidone), for example Kollidon® CL or CL-M available form BASF, poly(vinyl acetate) [PVAc], certain cellulose derivatives such as cellulose acetate, cellulose nitrate, alkyl cellulose such as ethyl cellulose, butyl cellulose, and isopropyl cellulose, cellulose acetate phthalate, unneutralized carboxymethyl cellulose shellac, ethylene-vinyl acetate copolymers, silicone polymer (e.g. dimethylsilicone), polyesters, polyurethanes, nylons, gums such as xanthan and carrageenan, polyethylene, and polypropylene. Acrylic copolymers may include carboxylic acid functionality which has not been neutralized at all or not been sufficiently neutralized to render the copolymer water soluble. Preferred acrylic copolymers are film-forming polymers. In certain embodiments, preferred acrylic copolymers may have weight average molecular weights at least about 20,000, more preferably at least about 50,000, and up to about 1,000,000, more preferably up to about 900,000. An example of a suitable, film-forming acrylic copolymer is Luvimer 30E, a 30% by weight solution in ethanol of a tert-butyl acrylate/ethyl acrylate/methacrylic acid copolymer available from BASF. The water-insoluble polymers may be prepared as dispersions (e.g., by emulsion polymerization) and may be stabilized with suitable emulsifiers. One useful PVAc emulsion, for example, is Kollicoat® SR 30 D, a 30 weight percent dispersion of PVAc in water stabilized with 2.7 weight percent PVP and 0.3% sodium lauryl sulfate. An example of an acrylic copolymer dispersion is Kollicoat® EMM 30 D, a 30% by weight aqueous dispersion of an ethyl acrylate : methyl methacrylate copolymer (weight ratio of ethyl acrylate to methyl methacrylate approximately 2 to 1) with a reported average molecular weight of about 800,000, available from BASF.

Other desired materials may be included in the film, such as, without limitation, surfactants, emulsifiers, plasticizers such as oils such as castor oil and mineral oil, glycerol, propylene glycol, and polyethylene glycol, clays, inert starch particles, cellulose, or other fillers, plastigels, waxes, and texture modifiers such as cold water swellable, physically modified and pregelatenized starches.

The film preferably also contains at least one functional material. As referred to herein, a “functional material” is a material providing aesthetic or decorative effects to the composition or an active material having a desired utility in the composition when it is used in cleansing. In some embodiments, the film can comprise a plurality of functional materials. In one embodiment, the compositions of the present invention comprise a plurality of first film fragments having a first functional material, and a plurality of second film fragments having a second functional material, wherein the second functional material differs from the first functional material.

A functional material may be included in the film to enhance the aesthetic appeal of the composition. For example, the functional material may be a colorant or pearlizing agent that is included to provide a desired color and/or color effect. Such a colorant may be selected from pigments, dyes, pearlescent colorants including pearlescent mica pigments, and water-soluble dyes contained within a water-insoluble polymer (such as polyethylene or another wax) to prevent bleeding. In various embodiments, the film comprises a formulation colorant, such as, for example, non-toxic water soluble dyes or pigment, such as, for example, metallic oxide “lakes.”

The functional material may also be an active material. Suitable examples of active materials include, without limitation, fragrances, essential oils, emulsifying agents, anti-microbial agents, vitamins, herbs, herbal extracts, exfoliating agents, deodorant actives, moisturizers, ultraviolet light absorbers and sunscreens, and combinations of these.

Other suitable active materials include materials that provide tactile sensation, help build lather, augment cleansing, and improve skin feel. Suitable examples of such materials include, without limitation, oatmeal, and fats and oils such as soybean oil, sunflower oil, meadowfoam seed oil, canola oil, various unsaturated mineral oils, and shea butter.

The films of the present invention may be made in a variety of ways, including methods among those known in the art for making films. In various embodiments, the film may be prepared by forming a slurry of the film materials. The slurry may be in an organic solvent or may be an aqueous slurry that may contain suitable organic solvents as well, such as ethanol, acetone, and ethyl acetate. The slurry is cast, drawn down, roll-coated, or otherwise applied in a layer on a releasable substrate and dried to form a sheet of film material. In one embodiment, the substrate material has a surface tension that allows the film slurry to spread substantially uniformly across the substrate surface, thereby avoiding formation of a destructive bond between the film and the substrate. Non-limiting examples of suitable substrates include glass, stainless steel, Teflon™ and polyethylene- or silicone-impregnated or -coated paper. Following casting, the film is then dried. Drying of the slurry can be carried out at elevated temperature, for example at a temperature of 60 to 100° C., with the aid of a drying oven, a drying terminal, a vacuum drier, or any other suitable drying equipment known in the art. In other embodiments, the film is made by extrusion of the film composition through a die, followed by cutting to a desired thickness, and drying. In other embodiments, the film may be made by solvent casting.

The film may comprise more than one layer. For example, a film can comprise a first layer comprising a polymer and one or more additional layers that provide a coating. The coating can be, for example, a shellac coating. A coating can comprise a layer on either or both sides of a polymer layer. Thus, in some embodiments, a process for making a multi-layered film can comprise forming a first layer of a film, such as a polymer layer, then coating the first layer with a second or subsequent layer of a coating material, for example shellac. In other embodiments, the film may comprise a layer having a first additive and another layer having another additive different from the first additive.

Shapes may be cut or punched from the film, or the film may be fragmented in a different manner to form fragments, flakes, or pieces of film that are incorporated into the carrier. In various embodiments, the film pieces exhibit perceivable contrast with the cleansing material. The perceivable contrast can be sensory contrast, such as optical contrast, tactile contrast, or contrast in fragrance. In some configurations, optical contrast can be color contrast, or it may be a difference in refractive index or reflective index. In some configurations, color contrast can be imparted by one or more colorants that comprise different components of the composition. In various embodiments, the present invention provides compositions comprising a plurality of film pieces in the composition material, wherein the film pieces are visibly discernable (that is, the film pieces have a different physical appearance from that of the cleansing base). Such characteristics include color, opacity, refractive index, reflective index, size, shape, and combinations of these.

The appearance of the film pieces, including their shape, color, and texture, may be suggestive, e.g., provide a visual cue, for a characteristic of composition. The film pieces may have different colors, shapes, or both to convey an impression of multiple benefits, either delivered by the film pieces or the composition material.

The film may have a thickness of preferably at least about 0.001 mm, more preferably at least about 0.01 mm, and up to about 0.8 mm, more preferably up to about 0.04 mm. In various embodiments, the size of the film pieces is not critical, and may be determined pursuant to any of a variety of criteria, including manufacturing convenience, affect on visual appearance, surface area, affect on texture, and combinations thereof. The film pieces may be present in a range of average sizes due to a variety of factors, including random variation in size, manufacturing tolerances, and intentional sizing or mixing of the pieces through sieving or similar means.

In some embodiments, the compositions may include film pieces having an aspect ratio of at least about 5:1. “Aspect ratio” of a film piece is the ratio of the diameter of the smallest imaginary sphere that can enclose the piece to the diameter of the largest imaginary sphere that can be completely inside the piece and tangent to the surfaces of the object. For example, the aspect ratio of a sphere is 1:1; in another example, the aspect ratio of a cylinder that is 2 inches (50.8 mm) long and ¼ inch (6.35 mm) in diameter is slightly over 8:1; in yet another example, a film piece that is 1 mil (25.4 microns) in thickness, 1 inch (25.4 mm) in length, and 1 inch (25.4 mm) wide has an aspect ratio of about 1414:1.

In some embodiments, the compositions of the present invention comprise film pieces having an aspect ratio of at least about 10:1. In various embodiments, the film pieces have an aspect ratio of about 5:1 to about 10,000: 1, about 10:1 to about 1,000: 1, or about 20:1 to about 100:1, or about 25:1 to about 35:1.

In certain embodiments, the film includes a polymer having a glass transition temperature or softening point high enough so that a film of the polymer is not sticky or soft at processing temperatures when making the composition. Preferably, film should include a film-forming polymer having a glass transition temperature of at least about 37° C., more preferably of at least about 60° C.

The film may have a film strength for certain embodiments such that the breaking strength of the film is at least about 800 psi. In certain embodiments, the film strength is at least about 1000 psi, or even at least about 2000 psi. In certain embodiments, the film strength is preferably between about 1000 and 1200 psi.

The film pieces may be included in various amounts in the cleansing material, or solid cleanser base. It is generally preferred to include at least about 0.001% by weight film pieces in the composition. This amount may be at least about 0.01 wt.%, more preferably at least about 0.05 wt.%, even more preferably at least about 0.1 wt.% of the composition, especially when the film is included for aesthetic attributes. Depending upon the utility of the film in the composition, the film may be included at much higher weight percents. The film may be included in amount of preferably up to about 5 wt.%, more preferably up to about 1 wt.% of the cleansing.

In various embodiments, the composition may have an opaque or a translucent or transparent cleanser base. In general, the cleanser base includes one or more cleansing agents such as soaps and surfactants. The cleansing agent or agents are included in an amount for effective cleansing ability. The cleanser base may preferably contain at least about 10% by weight soap and up to about 98% by weight of a soap. Suitable soaps include, without limitation, fatty carboxylates, including sodium salts, potassium salts, and amine soaps (e.g., triethanolamine soaps). Preferably, the soap includes soluble stearate, myristate, and/or cocoate salts.

The cleanser base may also include one or more surfactants. The surfactant may be selected from anionic, amphoteric, nonionic, and cationic surfactants.

Surfactants may preferably be included in the solid cleanser base in an amount of about 1%, by weight and may preferably be included in amounts of up to about 60% by weight.

The cleansing material may also include water and/or a humectant. Exemplary of these materials are propylene glycol, dipropylene glycol, glycerin, sorbitol, mannitol, xylitol, hexylene glycol, and the like. In certain embodiments, the moisture level of the cleansing base is preferably no greater than about 22% before batching.

The composition material may also include various fats and oils or skin conditioners for improved skin feel. Examples include, without limitation, soybean oil, sunflower oil, meadowfoam seed oil, canola oil, various unsaturated mineral oils, shea butter, silicone oils, waxes, fatty acids, esters, occlusive agents, petrolatum, waxes, high molecular weight alcohols, such as stearyl alcohol) and the like. Other emollients can also be in the composition. In certain embodiments, these materials can be used in relatively minor quantities, such as about 0.05 to about 3 to 4 wt % of each of these. Mixtures of conditioning agents can be used.

In certain embodiments, the composition material may also include vitamins and vitamin derivatives, such as vitamin E, vitamin E precursors, vitamin E acetate, botanical extracts, proteins, ceramides, antiirritants, α-hydroxy acids, β-hydroxy acids, sunscreen agents, tanning agents, skin lightening agents, skin firming agents, and combinations thereof.

In certain embodiments, the compositions may also include additives such as fragrances, antibacterial agents, moisturizers, humectants, colorants, chelating agents such as EDTA, and antimicrobial materials such as triclocarban, triclosan and the like.

In certain embodiments, the cleansing material may be made by combining the desired ingredients and then forming a solid composition material as chips or noodles. Additives used in minor amounts such as glycerin, silicone, antibacterials, fragrances, and colors can be mixed with the solid composition chip or noodles, or included in the chips or noodles themselves. The chips or noodles and film pieces are then mixed, either in an amalgamator or at any point thereafter in the standard toilet soap finishing line. Billets are formed, cut, and can be pressed into a desired size and shape. The extrusion cone may extrude the billet in a desired shape, such as a star shape, that will be used as cut (that is, without pressing).

It is preferred to add film to the composition material in the amalgamator for simpler processing and uniformity, or into a hopper at the upper stage of the vacuum prodder for minimizing deformation or degradation of the film pieces.

In other embodiments, the soap composition may be made by casting.

In one embodiment, a bar formed from the composition has a maximum depth of penetration of 60 mm as measured by bar hardness. Bar hardness is measured by using a penetrometer (Koehler Instrument Company). The depth of penetration (in mm) is measured after the 100 gram weighted needle is dropped freely into the bar surface from a point just above the bar and maintained for 5 seconds. The higher the depth of penetration, the softer the bar. Depth of penetration is measured immediately after the bar is removed from the packaging.

The composition is preferably used with water for cleansing. Cleansing with a composition having polymeric film pieces that include an active material and a water-soluble or water-dispersible polymer is a way of delivering an active material during cleansing.

### SPECIFIC EMBODIMENTS OF THE INVENTION

The invention is illustrated by the following examples. The examples are merely illustrative and do not in any way limit the scope of the invention as described and claimed. All parts are parts by weight unless otherwise noted. Various names of chemical components include those listed in the CTFA International Cosmetic Ingredient Dictionary (Cosmetics, Toiletry and Fragrance Association, Inc., 7^{th} ed. 1997).

### Example 1

A slurry is prepared by mixing together 80.65 parts by weight water, 3.9 parts by weight METHOCEL E15 (hydroxypropylmethyl cellulose, manufactured by Dow Chemical), 3.9 parts by weight METHOCEL E50 (hydroxypropylmethyl cellulose, manufactured by Dow Chemical), 4.4 parts by weight menthol, 2.6 parts by weight canola oil, 2 parts by weight titanium dioxide, 1.6 parts by weight Solka-floc 300 (manufactured by International Fiber Corp.), 0.25 parts by weight Tween 80 (polyoxyethylene sorbitan monooleate, manufactured by ICI Ltd.), 0.25 parts by weight glycerin, 0.25 parts by weight triacetin, and 0.2 parts by weight cornstarch. The slurry is drawn down onto silicone-coated film paper, then dried at 80° C. for about 15 minutes to provide a film with a thickness of about 1.6 mm. The film is cut into generally square fragments about 1.5 mm on each edge. Then, 0.1 parts by weight of the film fragments are mixed into a transparent soap base by adding the film fragments at the amalgamator. The soap is formed into a billet, cut, and pressed into a composition shape.

The white film squares retain their original shape after extrusion and processing into composition soap. The film squares provide a slight soft feel during use.

### Example 2

Example 1 is repeated, except that the slurry is prepared using 72.5 parts by weight water, 10 parts by weight METHOCEL® E5 (hydroxypropylmethyl cellulose, manufactured by Dow Chemical), 3 parts by weight METHOCEL® E50 (hydroxypropylmethyl cellulose, manufactured by Dow Chemical), 4.375 parts by weight menthol, 2.625 parts by weight canola oil, 1 part by weight titanium dioxide, 0.5 parts by weight Tween 80 (polyoxyethylene sorbitan monooleate, manufactured by ICI Ltd.), 2 parts by weight propylene glycol, and 4 parts by weight cornstarch, and the film fragments are incorporated into translucent soap base at a concentration of 0.06% by weight. Some elements may be broken but are still visually appealing.

During use of soap compositions that have different thicknesses of film flakes, the film pieces provide from no tactile perception to slight to moderate soft feel during use.

### Example 3

Example 2 is repeated, except that the 1 part by weight titanium dioxide is replaced by 1 part by weight Pigment Green 7.

### Example 4

Example 1 is repeated, using as the film-forming slurry a mixture of 73.95 parts by weight water, 9.55 parts by weight METHOCEL® E5 (hydroxypropylmethyl cellulose, manufactured by Dow Chemical), 6.875 parts by weight menthol, 4.125 parts by weight canola oil, 1 part by weight Pigment Green 7 (50%), 0.5 parts by weight Tween 80 (polyoxyethylene sorbitan monooleate, manufactured by ICI Ltd.), 0.1 parts by weight carageenan gum, and 4 parts by weight cornstarch,

### Example 5

Example 1 is repeated, using as the film-forming slurry a mixture of 73 parts by weight water, 10 parts by weight METHOCEL E5 (hydroxypropylmethyl cellulose, manufactured by Dow Chemical), 3 parts by weight METHOCEL E50 (hydroxypropylmethyl cellulose, manufactured by Dow Chemical), 2 parts by weight vitamin E acetate, 4 parts by weight vitamin E, 1 part by weight canola oil, 2 parts by weight D&C Red 30, 0.5 parts by weight Tween 80 (polyoxyethylene sorbitan monooleate, manufactured by ICI Ltd.), 0.5 parts by weight propylene glycol, and 4 parts by weight cornstarch. The soap containing the film fragments provides vitamin E and vitamin E acetate to the skin during use.

### Example 6

Example 1 is repeated, using as the film-forming slurry a mixture of 70 parts by weight water, 10 parts by weight METHOCEL® 5 (hydroxypropylmethyl cellulose, manufactured by Dow Chemical), 3 parts by weight METHOCEL® E50 (hydroxypropylmethyl cellulose, manufactured by Dow Chemical), 7.5 parts by weight triclosan, 4 parts by weight canola oil, 1 part by weight titanium dioxide, 0.5 parts by weight Tween 80 (polyoxyethylene sorbitan monooleate, manufactured by ICI Ltd.), 4 parts by weight propylene glycol, and 4 parts by weight cornstarch. The soap containing the film pieces provides an antibacterial action during use.

### Example 7

Example 1 is repeated, using as the film-forming slurry a mixture of 69.55 parts by weight water, 4 parts by weight cosmetic wax (manufactured by Dow Corning), 8.95 parts by weight poly(vinyl alcohol), 4 parts by weight cornstarch, 0.5 parts by weight titanium dioxide, 1 part by weight mineral oil, 1 part by weight PEG-600, 1 part by weight glycerin, and 10 parts by weight triclosan, The soap containing the film pieces provides an antibacterial action during use.

### Example 8

Example 1 is repeated, using as the film-forming slurry a mixture of 75.7 parts by weight water, 10 parts by weight METHOCEL E5 (hydroxypropylmethyl cellulose, manufactured by Dow Chemical), 3 parts by weight METHOCEL E50 (hydroxypropylmethyl cellulose, manufactured by Dow Chemical), 4 parts by weight modified cornstarch, 0.7 part by weight synthetic iron oxide red, 0. 1 part by weight titanium dioxide, 3 parts by weight mineral oil, 3 parts by weight PEG-600, and 0.5 part by weight Tween 80 (polyoxyethylene sorbitan monooleate, manufactured by ICI Ltd.). The soap containing the film pieces have a pleasing appearance.

### Example 9

Water soluble films were tested for dissolvability in the following manner:

10 pieces of 1/16″ film squares (Example 8) were placed on the palm of the hand, 3 drops of 32° C. tap water were added, the film was rubbed with the index finger of the opposite hand in a circular motion until the film completely dissolved. The number of rotations were counted. The mean # of rotations (n=7)=32; the range was 15 to 50.

### Example 10

In certain aspects of the invention, a visible particle is present in the composition that is not felt during washing or has a soft/gentle feel. In one embodiment, the composition can provide a softness/hardness rating of ≦2 according to the following test. In another embodiment, the composition can provide a gentle/roughness rating of ≦2.

Compositions with various particles were tested in the following manner: Panelists (a group of 7) were asked to rotate the composition in their hands as they normally would but to pay attention to the softness/hardness and gentleness/roughness of the particles. Panelists were asked to rate the feel based on the scales below:

Softness/Hardness Scale

0=not perceptible to touch during washing
1=very soft
2=soft
3=hard

4=very hard

Results for Soft/Hardness

| | bar with film | bar with oat flour | bar with polyethylene |
|---|---|---|---|
| range | 0 to 1 | 2 to 4 | 2 to 4 |
| Mean | 0.3 | 2.9 | 3.0 |

Gentle/Rough Scale
0=not perceptible to touch during washing
1=very gentle
2=gentle
3=rough

4=very rough

Results for Gentle/Roughness

| | bar with film | bar with oat flour | bar with polyethylene |
|---|---|---|---|
| range | 0 to 1 | 2 to 4 | 2 to 4 |
| Mean | 0.3 | 2.7 | 2.7 |

### Example 11

A composition may be made with the types and amounts of ingredients listed in Table A, using the following method. Mix and heat the water, cocoamidopropyl betaine, and humectants, including glycerin, to 80° C. to 110° C. Once at temperature, the mixture was charged with the gellant (DBS) and mixing is continued until the batch is clear. Once the gellant is fully dissolved, the sodium chloride is added. Once the sodium chloride is dissolved, the acids are added to the batch. Upon complete dissolution of the acids, the sodium hydroxide is added to the batch to react with the acids and form the soap. Temperature of the batch is maintained at less than 105° C. The batch is then neutralized with the coconut acid. After neutralization, the surfactants are mixed in until uniform. Following the surfactants at a temperature less than 90° C., the remaining minor (optional) ingredients are added: color, skin feel agents, and fragrance, and mixed until uniform. The film is added last into the mixing batch and is uniformly dispersed. The temperature incorporated was 70-75° C. The molten soap is then poured into a mold and allowed to cool to a solid form.

TABLE A

| | |
|---|---|
| Propylene Glycol | 16 |
| Stearic Acid | 14 |
| 70% Sodium Laureth Sulfate | 12 |
| 30% Cocoamidopropyl Betaine | 8.73 |
| 50% Sodium Hydroxide | 7.44 |
| Myristic Acid | 7.3 |
| Water | 5.98 |
| Disodium Lauryl Sulfosuccinate | 4.5 |
| Glycerin | 4 |
| Sucrose | 4 |
| Sodium Lauryl Sulfate | 4 |
| Coconut Acid | 3.5 |
| Dipropylene Glycol | 2 |
| Cocamide MEA | 2 |
| Dimethicone polyol | 2 |
| (DC 190 from Dow Corning) | |
| Sodium Chloride | 1 |
| Fragrance | 1 |
| Dibenzylidene sorbitol | 0.25 |
| Ethanol | 0.2 |
| Film | 0.1 |
| Colorant | 0.0001 |

### Example 12

A soap composition made according to the process of Example 11 using the ingredients listed in Table A appeared translucent with visible film pieces. The composition appearance was stable after 2 months room temperature aging. The moisture content was measured to be 17.6% using Karl Fischer. Target moisture was 18.5%.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A composition comprising a cleanser base and a plurality of film pieces, wherein the composition has a moisture content of about 22% or less.

2. The composition of claim 1, wherein the film pieces comprise a film-forming polymer.

3. The composition of claim 2, wherein the film-forming polymer comprises a polymer selected from the group consisting of water-soluble polymers and water-dispersible polymers.

4. The composition of claim 3, wherein the water-soluble polymer is selected from the group consisting of methyl cellulose, ethyl methyl cellulose, hydroxyalkyl celluloses, hydroxyalkyl alkyl celluloses, carboxyalkyl celluloses, and combinations thereof.

5. The composition of claim 1, wherein the film pieces comprise hydroxypropylmethyl cellulose.

6. The composition of claim 1, wherein the film pieces comprise one or more of a pigment, a dye, a pearlescent colorant, or a water-soluble dye contained within a water-insoluble material.

7. The composition of claim 1, wherein the film pieces comprise a functional material.

8. The composition of claim 7, wherein the functional material is an active agent.

9. The composition of claim 7, wherein the functional material is selected from the group consisting of essential oils, emulsifying agents, anti-microbial agents, vitamins, herbs, herbal extracts, exfoliating agents, deodorant actives, moisturizers, sunscreens, ultraviolet light absorbers, fragrances, emollients, skin conditioners, and combinations thereof.

10. The composition of claim 2, wherein the film-forming polymer comprises a water-insoluble polymer.

11. The composition of claim 1, wherein the film pieces comprise a water-soluble filler.

12. The composition of claim 1, wherein the film pieces comprise a layer having a first additive and at least one more layer having another additive different from the first additive.

13. The composition of claim 1, wherein the film pieces and the cleanser base differ in their respective refractive indices.

14. The composition of claim 1, wherein the film pieces and the cleanser base have about the same refractive indices.

15. The composition of claim 1, wherein the film pieces have an aspect ratio of at least about 5:1.

16. The composition of claim 1, wherein the plurality of film pieces has a softness/hardness rating of ≦2 and/or a gentleness/roughness rating of ≦2.

17. A bar comprising a cleanser base and a plurality of pieces of film.

18. The bar of claim 16, wherein the film comprises a polymer selected from the group consisting of water-soluble polymers and water-dispersible polymers.

19. The bar of claim 17, wherein the polymer further comprises a water-insoluble polymer.

20. A method of making a composition, comprising the steps of
combining pieces of film with a cleansing material to make a cleansing mixture, and forming the mixture into a product of desired shape and size.

21. A method of delivering an active material during cleansing, comprising cleansing with water and a composition, wherein the composition comprises a plurality of film pieces, said film pieces comprising an active material and a polymer selected from water-soluble polymers, water-dispersible polymers, and combinations thereof.
